(19) United States
(12) Patent Application Publication
(10) Pub. No.: US 2016/0289300 A1
(43) Pub. Date: Oct. 6, 2016

(54) METHOD OF MANUFACTURING INTRAVENOUS IMMUNOGLOBULIN FROM FRACTION III

(71) Applicant: Kieu Hoang, Agoura Hills, CA (US)

(72) Inventor: Kieu Hoang, Agoura Hills, CA (US)

(21) Appl. No.: 15/090,025

(22) Filed: Apr. 4, 2016

Related U.S. Application Data
(60) Provisional application No. 62/142,212, filed on Apr. 2, 2015.

(51) Int. Cl. C07K 16/06

(52) U.S. Cl. CPC C07K 16/065; C07K 2317/51; C07K 2317/515; C07K 2317/21; C07K 2317/73

(57) ABSTRACT

The present subject matter is directed to a method of manufacturing purified IVIG from Fraction III of plasma, comprising re-constituting a Fraction III paste in a buffer; adjusting the pH and temperature; adding ethanol and then gradually lowering the temperature; centrifuging and filtering the supernatant; ultra-filtrating to remove alcohol; undergoing weak anion exchange chromatography; ultra-filtrating to reach a desired protein concentration; aseptic filtrating; nano filtrating for virus removal; and incubating at low pH for virus inactivation to obtain a resulting Fraction III suspension comprising purified IVIG. The present subject matter is directed to IVIG having 14 newly-found proteins, namely KH 26, KH 27, KH 28, KH 29, KH 30, KH 31, KH 32, KH 33, KH 39, KH 40, KH 41, KH 42, KH 43, and KH 44 for both liquid and lyophilized form.

### RELATED APPLICATIONS

The present patent application claims priority to provisional U.S. Patent Application No. 62/142,212 filed Apr. 2, 2015, which was filed by the inventor hereof and are incorporated by reference herein in its entirety.

### TECHNICAL FIELD

The present subject matter relates to intravenous immunoglobulin (IVIG) produced from Fraction III of plasma and applications thereof, particularly for the treatment and prevention of Hepatitis C virus. In particular, the present subject matter is associated with the manufacturing process of IVIG from Fraction III, which contains 14 newly-found proteins, namely KH 26, KH 27, KH 28, KH 29, KH 30, KH 31, KH 32, KH 33, KH 39, KH 40, KH 41, KH 42, KH 43, and KH 44 for both liquid and lyophilized forms.

### BACKGROUND

Immunoglobulin G (IgG) is a type of antibody and is a protein complex composed of four peptide chains—two identical heavy chains and two identical light chains arranged in a Y-shape typical of antibody monomers. Each IgG has two antigen binding sites. Representing approximately 75% of serum antibodies in humans, IgG is the most common type of antibody found in the circulatory system. IgG molecules are typically created and released by plasma B cells. Antibodies are major components of humoral immunity. IgG is the main type of antibody found in blood and extracellular fluid, allowing it to control infection of body tissues. IgG protects the body from infection by binding many kinds of pathogens, such as viruses, bacteria, and fungi. In the plasma-derived industry, IgG is usually purified from human plasma from Fraction II. However, a certain percentage of IgG may be precipitated from Fraction III paste, which includes 34 existing and newly-found proteins.

### SUMMARY

In an embodiment of the present subject matter, 34 existing and newly-found proteins were found to be present in the Fraction III extracted from plasma. The 14 newly-found proteins and 20 existing proteins can be processed and purified to make an intravenous solution of Immunoglobulin (IVIG). In an embodiment, up to 20% of IVIG administered to a human can be that purified from Fraction III. In an embodiment, with the addition of the newly-found proteins, the intravenous solution of immunoglobulin not only stops replication of Hepatitis C Virus, but also prevents Hepatitis C virus infection.

Among the 34 proteins, 14 are newly-found proteins according to the present subject matter. According to the present subject matter, IgG may be recovered from Fraction III paste, which includes these 14 newly-found proteins for intravenous injection against Hepatitis C virus. The recovered product not only stops replication of the Hepatitis C virus, but also kills the Hepatitis C Virus. Thus, the Hepatitis C virus infection may be eradicated and prevented from the world.

In an embodiment, the present subject matter is directed to a method of manufacturing purified IVIG from Fraction III of plasma, comprising the steps:
a) re-constituting a Fraction III paste in a sodium chloride sodium citrate buffer to obtain a reconstituted Fraction III;
b) adjusting the pH of the reconstituted Fraction III to a range from 3 to 8 and a temperature from −5° C. to 30° C. to obtain an adjusted Fraction III;
c) adding ethanol to the adjusted Fraction III up to 10-40% and then gradually lowering the temperature to −10° C. to 20° C.;
d) centrifuging to collect a supernatant;
e) filtering the supernatant with a 10CP+90SP filter to obtain a resulting solution;
f) ultra-filtrating the resulting solution to remove alcohol;
g) undergoing weak anion exchange chromatography to collect a flow-through solution;
h) ultra-filtrating the flow-through solution to reach a desired protein concentration;
i) aseptic filtrating the flow-through solution;
j) nano filtrating the flow-through solution for virus removal with a 20 nm filter; and
k) incubating the flow-through solution at low pH for virus inactivation to obtain a resulting Fraction III suspension comprising purified IVIG.

### BRIEF DESCRIPTION OF THE DRAWINGS

FIG. 1 is a flow chart depicting methods of processing IVIG from Fraction III.

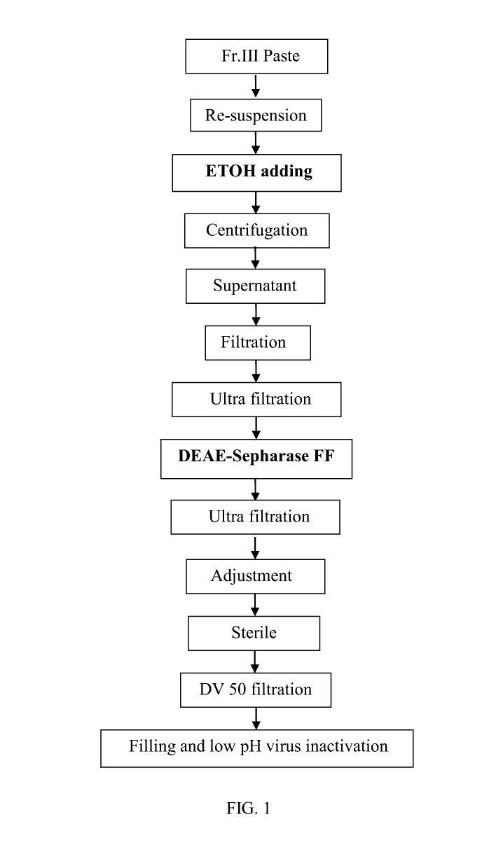

FIGS. 2A-C show a comparison of regular GammaRAAS (FIG. 2A), Fraction III IVIG first batch (FIG. 2B), and Fraction III IVIG second batch (FIG. 2C) by 2D electropherosis. In GammaRAAS, the known proteins from the Fraction II paste were found, but contain none of the 14 newly-found proteins in WIG from Fraction III.

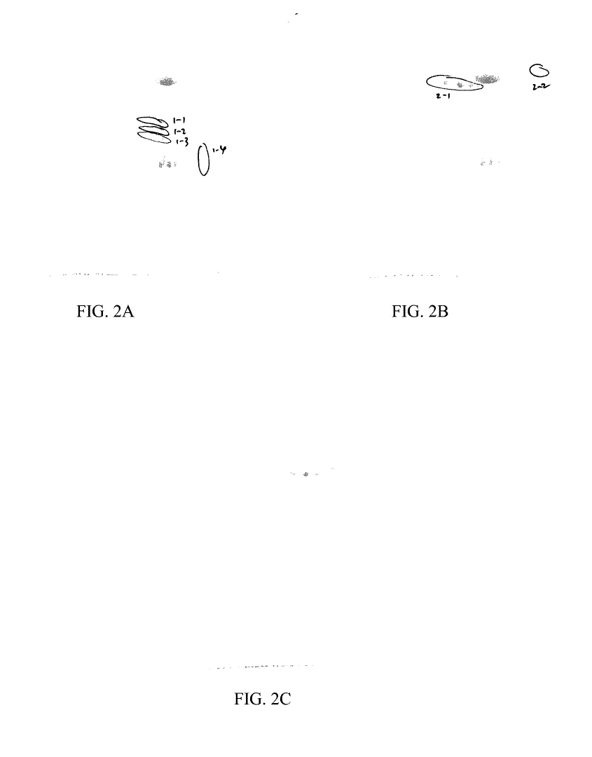

### DETAILED DESCRIPTION

Unless defined otherwise all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language; however, it will be understood by one of skill in the art, that in some specific instances, an embodiment can alternatively be described using the language “consisting essentially of or “consisting of.”

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

An embodiment of the present subject matter is directed to a method of manufacturing purified IVIG from Fraction III of plasma, comprising the steps:
a) re-constituting a Fraction III paste in a sodium chloride sodium citrate buffer to obtain a reconstituted Fraction III;
b) adjusting the pH of the reconstituted Fraction III to a range from 3 to 8 and a temperature from −5° C. to 30° C. to obtain an adjusted Fraction III;
c) adding ethanol to the adjusted Fraction III up to 10-40% and then gradually lowering the temperature to −10° C. to 20° C.;
d) centrifuging to collect a supernatant;
e) filtering the supernatant with a 10CP+90SP filter to obtain a resulting solution;
f) ultra-filtrating the resulting solution to remove alcohol;
g) undergoing weak anion exchange chromatography to collect a flow-through solution;
h) ultra-filtrating the flow-through solution to reach a desired protein concentration;
i) aseptic filtrating the flow-through solution;
j) nano filtrating the flow-through solution for virus removal with a 20 nm filter; and
k) incubating the flow-through solution at low pH for virus inactivation to obtain a resulting Fraction III suspension comprising purified IVIG.

In an embodiment, the Fraction III paste may be obtained by a Cohn ethanol fractionation method. In an embodiment, the resulting Fraction III suspension further comprises proteins KH 26, KH 27, KH 28, KH 29, KH 30, KH 31, KH 32, KH 33, KH 39, KH 40, KH 41, KH 42, KH 43, and KH 44. In an embodiment, the adjusted Fraction III is gently mixed with cold alcohol to reach a final concentration of 10-40% and temperature of −10° C. to 20° C. The ethanol may be added to the adjusted Fraction III up to 18-20% and the temperature may be gradually lowered to −7° C. to −5° C.

In an embodiment of the present subject matter, the flow-through solution is ultra filtered with a 10 K cutoff membrane. The method of manufacturing purified IVIG from Fraction III may further comprise adjusting the protein concentration and pH of the flow-through solution. In an embodiment, the weak anion exchange chromatography is conducted using DEAE sepharose FF as the chromatopgraphy medium. In an embodiment, the aseptic filtration is 0.22 aseptic filtration.

An embodiment of the present subject matter is directed to a method of manufacturing purified IVIG from Fraction III of plasma wherein the resulting Fraction III suspension is obtained by filling and low pH incubation at pH 4 for 21 days at 25° C. for virus inactivation.

Furthermore, an embodiment of the present subject matter is directed to a method of stopping replication of a Hepatitis C virus in a patient comprising administering the purified IVIG obtained from the method of manufacturing purified IVIG from Fraction III of plasma to a patient in need thereof. An embodiment of the present subject matter is directed to a method of killing a Hepatitis C virus in a patient comprising administering the purified IVIG obtained from the method of manufacturing purified IVIG from Fraction III of plasma to a patient in need thereof. An embodiment of the present subject matter is directed to a method of preventing infection of a Hepatitis C virus in a patient comprising administering the purified IVIG obtained from the method of manufacturing purified IVIG from Fraction III of plasma to a patient in need thereof.

Moreover, an embodiment of the present subject matter is directed to a method of treatment for a patient in need thereof comprising administering the purified IVIG obtained from the method of manufacturing purified IVIG from Fraction III of plasma to the patient, wherein the purified IVIG transforms or repairs damaged and sick cells to become healthy cells, wherein the purified IVIG protects cellular alterations, and wherein the purified IVIG sends signals to a body to produce new cells that are healthy, thereby preventing the new cells from being affected by intracellular and extracellular damaging signals.

Further, an embodiment of the present subject matter is directed to a purified IVIG produced according to the method of manufacturing purified IVIG from Fraction III of plasma. The purified IVIG may further comprise proteins KH 26, KH 27, KH 28, KH 29, KH 30, KH 31, KH 32, KH 33, KH 39, KH 40, KH 41, KH 42, KH 43, and KH 44. The purified IVIG may be in liquid form or lyophilized form.

An embodiment of the present subject matter is directed to an IVIG comprising up to 20% of the purified IVIG produced according to the method of manufacturing purified IVIG from Fraction III of plasma.

### In Vitro Testing

IVIG purified from Fraction III, with 14 newfound proteins, was tested by one of the top clinical research organizations in China, which concluded that Fraction III IVIG, Code name AFCC RAAS6, has the ability to stop replication and kill the Hepatitis C virus. Tables 1 and 2 display the quantification test results.

TABLE 1
Quantification Test Results for HBV

| | Quantification Test |
|---|---|
| Sample Name | Results (IU/ml) |
| 500 HBV + AFOD-KH | 8.18E+1 |
| 500 HBV + AFCC-RAAS-2 | <2.00E+1 |
| 500 HBV + AFCC-RAAS-6 | 5.04E+1 |
| 500 HBV + AFCC-RAAS-8 | <2.00E+1 |
| 500 HBV + Negative Plasma | 4.41E+1 |

Note:
The detection limit for HBV quantification is 2.00E+1 IU/ml.

TABLE 2
Quantification Test Results for HCV for IVIG from Fraction III

| | Quantification Test |
|---|---|
| Sample Name | Results (IU/ml) |
| 105 HCV + AFOD-KH | 2.8E+04 |
| 105 HCV + AFCC-RAAS-2 | 8.1E+05 |
| 105 HCV + AFCC-RAAS-6 | <25.0 |
| 105 HCV + AFCC-RAAS-8 | 2.4E+05 |
| 105 HCV + Negative Plasma | 2.11E+3 |

Note:
The detection limit for HCV quantification is 25 IU/ml.

### EXAMPLES

According to an embodiment of the present subject matter, . . .

### Testimonials

An embodiment . . .

With the information contained herein, various departures from precise descriptions of the present subject matter will be readily apparent to those skilled in the art to which the present subject matter pertains, without departing from the spirit and the scope of the below claims. The present subject matter is not considered limited in scope to the procedures, properties, or components defined, since the preferred embodiments and other descriptions are intended only to be illustrative of particular aspects of the presently provided subject matter. Indeed, various modifications of the described modes for carrying out the present subject matter which are obvious to those skilled in chemistry, biochemistry, or related fields are intended to be within the scope of the following claims.

I claim:

1. A method of manufacturing purified IVIG from Fraction III of plasma, comprising the steps:
a) re-constituting a Fraction III paste in a sodium chloride sodium citrate buffer to obtain a reconstituted Fraction III;
b) adjusting the pH of the reconstituted Fraction III to a range from 3 to 8 and a temperature from −5° C. to 30° C. to obtain an adjusted Fraction III;
c) adding ethanol to the adjusted Fraction III up to 10-40% and then gradually lowering the temperature to −10° C. to 20° C.;
d) centrifuging to collect a supernatant;
e) filtering the supernatant with a 10CP+90SP filter to obtain a resulting solution;
f) ultra-filtrating the resulting solution to remove alcohol;
g) undergoing weak anion exchange chromatography to collect a flow-through solution;
h) ultra-filtrating the flow-through solution to reach a desired protein concentration;
i) aseptic filtrating the flow-through solution;
j) nano filtrating the flow-through solution for virus removal with a 20 nm filter; and
k) incubating the flow-through solution at low pH for virus inactivation to obtain a resulting Fraction III suspension comprising purified IVIG.

2. The method of claim 1, wherein the Fraction III paste is obtained by a Cohn ethanol fractionation method.

3. The method of claim 1, wherein the resulting Fraction III suspension further comprises proteins KH 26, KH 27, KH 28, KH 29, KH 30, KH 31, KH 32, KH 33, KH 39, KH 40, KH 41, KH 42, KH 43, and KH 44.

4. The method of claim 1, wherein the adjusted Fraction III is gently mixed with cold alcohol to reach a final concentration of 10-40% and temperature of −10° C. to 20° C.

5. The method of claim 1, wherein ethanol is added to the adjusted Fraction III up to 18-20% and then the temperature is gradually lowered to −7° C. to −5° C.

6. The method of claim 1, wherein the flow-through solution is ultra filtered with a 10 K cutoff membrane.

7. The method of claim 1, further comprising adjusting the protein concentration and pH of the flow-through solution.

8. The method of claim 1, wherein the weak anion exchange chromatography is conducted using DEAE sepharose FF.

9. The method of claim 1, wherein the aseptic filtration is 0.22 μm aseptic filtration.

10. The method of claim 1, wherein the resulting Fraction III suspension is obtained by filling and low pH incubation at pH 4 for 21 days at 25° C. as the virus inactivation.

11. A method of stopping replication of a Hepatitis C virus in a patient comprising administering the purified IVIG obtained from the method of claim 1 to a patient in need thereof.

12. A method of killing a Hepatitis C virus in a patient comprising administering the purified IVIG obtained from the method of claim 1 to a patient in need thereof.

13. A method of preventing infection of a Hepatitis C virus in a patient comprising administering the purified IVIG obtained from the method of claim 1 to a patient in need thereof.

14. A method of treatment for a patient in need thereof comprising administering the purified IVIG obtained from the method of claim 1 to the patient,
wherein the purified IVIG transforms or repairs damaged and sick cells to become healthy cells,
wherein the purified IVIG protects cellular alterations, and
wherein the purified IVIG sends signals to a body to produce new cells that are healthy, thereby preventing the new cells from being affected by intracellular and extracellular damaging signals.

15. A purified IVIG produced according to the method of claim 1.

16. The purified IVIG of claim 15 furthering comprising proteins KH 26, KH 27, KH 28, KH 29, KH 30, KH 31, KH 32, KH 33, KH 39, KH 40, KH 41, KH 42, KH 43, and KH 44.

17. The purified IVIG of claim 15, wherein the purified IVIG is in liquid form.

18. The purified IVIG of claim 15, wherein the purified IVIG is in lyophilized form.

19. An IVIG comprising up to 20% of the purified IVIG produced according to the method of claim 1.
